(19) United States
(12) Patent Application Publication
(10) Pub. No.: US 2026/0199432 A1
(43) Pub. Date: Jul. 16, 2026

(54) COMPOUND WITH ANTI-METASTATIC EFFECT

(71) Applicant: ONCOGREEN THERAPEUTICS S.A., Lugano (CH)

(72) Inventors: Renzo DAL MONTE, Brendola (Vicenza) (IT); Andrea CARPI, Brendola (Vicenza) (IT)

(21) Appl. No.: 19/127,850

(22) PCT Filed: Nov. 7, 2023
(86) PCT No.: PCT/IB2023/061227

§ 371 Date: May 7, 2025

(30) Foreign Application Priority Data
Nov. 8, 2022 (IT) 102022000023031

(51) Int. Cl. A61K 38/17; A61K 9/00; A61P 35/04; C12N 15/82

(52) U.S. Cl. CPC A61K 38/1796; A61K 9/0019; A61P 35/04; C12N 15/8202; C12N 15/8257; C12N 2800/10

(57) ABSTRACT

The present invention relates to the medical use of a compound in the treatment of tumor metastases.

### TECHNICAL FIELD OF THE INVENTION

The present invention finds application in the medical field, and in particular in the therapy of tumors through the stimulation of the immune system.

### BACKGROUND ART

Several small molecules of both natural and synthetic origin have been used in the therapy of tumors. These molecules carry out their activity on tumor cells through the inhibition of particular signaling pathways or show cytotoxic activity (mitosis inhibitors, inhibitors of microtubule formation, inhibitors of DNA repair systems, etc.). However, all these small molecules are not specific for tumor cells and have a high systemic toxicity, causing serious side effects up to the appearance of serious diseases with a poor prognosis in patients. In recent decades, in order to try to reduce or eliminate this problem, the use of monoclonal antibodies directed towards specific receptors on tumor cells has been introduced into the therapy of cancer patients. Despite some success of this approach, problems related to the specificity of the therapies remain. In fact, the targets of monoclonal antibodies used in tumor therapy are not present only in the tumor cells, but also in other healthy body tissues/organs. Therefore, also in this case the therapy causes serious side effects in patients. Despite this, in the last decade new generations of monoclonal antibodies linked to cytotoxic molecules have continued to be produced for the purpose of increasing the therapeutic efficacy thereof, although at the same time non-specific damage to patients' non-diseased tissues is also increasing. More recently, a new immunotherapy-based approach to the treatment of tumors has been introduced. In particular, the patient's T lymphocytes are collected and genetically modified to increase the ability thereof to specifically recognize and kill tumor cells. This strategy is known as CAR-T (Chimeric Antigen Receptor T-cell). However, the therapy is very expensive, depends significantly on the features of the facility where it is practiced, and last but not least, the success thereof is strictly dependent on the features of the patient treated. The CAR-T therapy also shows non-negligible side effects due to the fact that the target molecules recognized by the genetically modified T lymphocytes are not present only on tumor cells. Recently and still in the embryonic stage, a new strategy which allows stimulating macrophages, a particular class of cells of the immune system, has been suggested. The goal is to make macrophages acquire anti-tumor features and make them capable of specifically activating the other components of the immune system against the aberrant cells.

International patent application WO 2018/069831 describes the use of the beta subunit of the follicle stimulating hormone receptor, as well as a recombinant subunit, for treating and diagnosing tumors; the document does not describe the use of the subunit for treating and/or preventing metastases.

### SUMMARY OF THE INVENTION

The present invention is based on having surprisingly found a compound which is capable of reprogramming macrophages in an anti-tumor sense.

In particular, it has surprisingly been found that such a compound can be used for treating and/or preventing the formation of metastases resulting from a primary tumor.

### OBJECT OF THE INVENTION

A first object of the present invention is represented by the medical use of a compound for treating and/or preventing tumor metastases.

In a preferred aspect, such a compound for treating and/or preventing tumor metastases is recombinant (ABRβ).

In a particular aspect of the invention, the described medical use for treating and/or preventing tumor metastases occurs by means of the stimulation of the immune system.

In another aspect of the invention, the described medical use for treating and/or preventing tumor metastases occurs, because the tumor-associated macrophages M2 (TAM-M2) are reprogrammed to tumor-associated macrophages M1 (TAM-M1).

In a second object, the present invention describes pharmaceutical preparations comprising said compound.

In a further object, a method for treating and/or preventing tumor metastases is described, comprising administering a pharmaceutically effective amount of the compound of the invention to a subject in need thereof.

### BRIEF DESCRIPTION OF THE DRAWINGS

FIG. 1 shows the effect of ABRβ1 1 on macrophage IL10 production: anti-tumor macrophages M1, pro-tumor macrophages M2, untreated NT, lipopolysaccharide LPS.

FIG. 2 shows the effect of ABRβ1 on macrophage IL12 production: anti-tumor macrophages M1, pro-tumor macrophages M2, untreated NT, lipopolysaccharide LPS.

FIG. 3 shows the effect of ABRβ1 on survival in a syngeneic mouse model of melanoma.

### DETAILED DESCRIPTION OF THE INVENTION

In accordance with a first object of the present invention, the medical use of a compound for treating and/or preventing tumor metastases is described.

According to a preferred aspect of the invention, such a compound is represented by the β subunit of follicle stimulating hormone (FSH) (SEQ. ID NO: 1) or is a B subunit of recombinant follicle stimulating hormone (FSH), described herein for treating and/or preventing tumor metastases.

### Recombinant Compound (ABRβ)

For the purposes of the present invention, the term “ABRβ” refers to the beta subunits of human follicle stimulating hormone (FSH) obtainable by means of the use of a biotechnological platform, described below.

“ABRβ1” means a specific beta subunit of human follicle stimulating hormone (FSH) obtained by means of the use of the biotechnological platform for the production of Nicotiana benthamiana in plant cells in accordance with the present invention.

In particular, such a subunit is characterized by the amino acid sequence corresponding to SEQ. ID NO: 2.

HHHHHHNSCELTNITIAIEKEECRFCISINTTWCAGYCYTRDLVY
KDPARPKIQKTCTFKELVYETVRVPGCAHHADSLYTYPVATQCHC
GKCDSDSTDCTVRGLGPSYCSFGEMKEKDEL

The biotechnological platform for preparing the β subunit of human follicle stimulating hormone (FSH) comprises the use of transformed cells.

According to an aspect of the present invention, such cells can be plant cells.

According to a preferred aspect of the invention, such cells are for example Nicotiana benthamiana (ABRβ1) cells.

In particular, the ABRβ1 recombinant compound of the present invention is glycosylated to asparagine residues 13 and 30 of the mature protein.

More in detail, the glycosylation sites comprise branched structures of mannose residues.

The mannose residues are in an overall number of about 45-75, preferably about 50-70 and even more preferably about 58-62, where they can be 60 or 61.

Each glycosylation site comprises two N-acetylglucosamine residues and a branched structure of mannose residues.

In particular, each branched structure comprises 29, 30 or 31 mannose residues.

Each mannose residue can comprise phosphorylations or sulfurylations or methylations.

Moreover, the polysaccharide portions can be linked to molecules comprising phenolic groups.

Molecules comprising phenolic groups are characteristic of plant cells.

In particular, such phenolic groups are those typical of Nicotiana benthamiana plant cells.

In particular, the process for preparing the ABRβ1 subunit comprises the modification at the C-terminus with a KDEL sequence and at the N-terminus with a 6-histidine tail (His-tag) of the β subunit of human follicle stimulating hormone (FSHB) (SEQ. ID NO: 1).

The platform allowing the preparation of the subunit of the present invention is described for example in international patent application WO 2018/069831.

More in detail, the process for preparing a recombinant form of the β subunit follicle stimulating hormone (FSH) comprises the steps of:
I) obtaining an appropriate vector transformed with a plasmid comprising the sequence corresponding to SEQ. ID NO. 3;
II) transforming the plant cells with the vector of step I);
III) selecting the transformed plant cells;
IV) culturing the stable plant cells;
V) preparing a cell extract;
VI) purifying the compound.

In a preferred aspect, the vector of step I) is represented by Agrobacterium tumefaciens.

The sequence used for the transformation corresponds to SEQ. ID NO. 3:

GAATTCAACAATGGCTACTCAGAGAAGGGCTAACCCATCTTCTCT
TCACCTGATTACCGTGTTCTCTCTGCTTGTGGCTGTGGTGTCTGC
TGAGGTGTTCCATCATCACCATCATCACAATTCTTGCGAGCTGAC
CAACATCACCATTGCTATCGAGAAAGAAGAGTGCAGGTTCTGCAT
CAGCATCAACACTACTTGGTGCGCTGGTTACTGCTACACCAGGGA
TCTTGTGTACAAGGATCCTGCTAGGCCTAAGATCCAAAAGACCTG
CACCTTCAAAGAGCTGGTTTACGAGACTGTTAGGGTGCCAGGTTG
TGCTCATCATGCTGATTCTCTGTACACCTACCCTGTTGCTACTCA
GTGCCATTGCGGTAAGTGCGATAGCGATTCTACTGATTGCACCGT
GAGAGGTCTGGGACCTTCTTACTGTTCTTTCGGTGAGATGAAAGA
AAAGGATGAGCTGTAGTCTAGA

For the purposes of the present invention, step II) comprises the transformation of Nicotiana benthamiana plant cells.

In particular, in step II) the transformation is carried out for 48 hours of co-culture in the dark at about 25° C. and under constant stirring.

The cells are then selected.

Preferably, a selection medium is used in step III) comprising: MS added with 0.9% w/v agar and antibiotics.

In a preferred aspect, carbenicillin and kanamycin, more preferably 250 mg/L carbenicillin and 100 mg/L kanamycin, are used for such a purpose.

In a preferred aspect of the invention, in step IV) the cells, possibly of Nicotiana benthamiana, are cultured in suspension.

In another preferred aspect, the cultivation includes an initial inoculation of plant cells, possibly of Nicotiana benthamiana, equal to 10% of the final culture volume.

The cell culture is incubated in MS medium (Murashige 1962) added with sucrose, naphthalene-acetic acid (NAA) and kinetin for a period of 15 days at a temperature of 24-27° C. and maintaining an aeration of 50-100 mbar.

Moreover, subcultures are set up every 7 days, transferring an aliquot of cell suspension into the fresh medium.

The cells are incubated under stirring, in the dark and at a constant temperature of 25° C.

In accordance with the present invention, step V) comprises the use of an extraction buffer comprising: 50 mM Na₂HPO₄, 150 mM NaCl, 20 mM citric acid, 40 mM ascorbic acid, 5 mM EDTA, 1 mM PMSF, 0.05% (v/v) Tween-20, pH 6.5 added with 1% (w/v) XAD-4 and 1% (w/v) polyvinylpolypyrrolidone (PVPP).

Ammonium sulfate is then added to the extract until a saturation concentration of 70% is obtained, incubated at 4° C. for 1 hour under constant stirring.

The precipitate is then recovered by centrifugation and resuspended in an IMAC buffer.

The preparation is centrifuged and filtered.

The solution thus obtained is purified in a step VI) through column passages.

In particular, the solution is loaded on an IMAC chromatography column.

Preferably, an Ni Sepharose 6 FF column is used.

The fractions of interest are then gathered and loaded on a desalting column.

Preferably, a Sephadex G-25 Medium column is used.

The fractions of interest are then gathered and loaded on an ion exchange chromatography column.

Preferably, an SP Sepharose HP column is used.

During purification, the absorbance is monitored at 280 and 254 nm.

As described above, the ABRβ1 subunit is obtained biotechnologically (recombinantly) from Nicotiana benthamiana plant cell culture in suspension.

According to alternative aspects, the ABRβ subunit of the present invention can be obtained biotechnologically in other cells, for example in mammalian, yeast, bacterial or other plant cells.

In particular, cells of Daucus carota, Oryza sativa, Glycine max, Maize, etc., can be used among the plant cells.

For the purposes of the present invention, the term “treatment” of tumor metastases means a therapeutic protocol in a patient having a primary tumor with or without the presence of metastases.

In a preferred aspect of the invention, such primary tumors are represented by melanoma, breast, ovary, sarcomas, pancreas, kidney, stomach, lung tumors, neuroblastoma (even in pediatric age).

For the purposes of the present invention, the term “tumor metastases” means tumors, in particular solid, secondary tumors originating from a primary tumor.

In a preferred aspect of the invention, such metastases are metastases originating from melanoma.

In an even more preferred aspect of the invention, the metastases are lung, peritoneum, brain, kidneys, liver metastases.

In a further aspect, the present invention describes the medical use of the compound, possibly recombinant (ABRβ), for preventing metastases.

The compounds described by the present invention are thus anti-metastatic.

This means that in the patient with a primary tumor, the recombinant compound (ABRβ) of the present invention not only causes the regression of metastases already formed by the primary tumor (therapeutic effect), but can also prevent the formation thereof.

According to a preferred aspect of the present invention, the medical use of a compound of the invention for treating and/or preventing tumor metastases is described, in which such a treatment and/or prevention occur by means of stimulation of the immune system.

In another aspect of the invention, the medical use for treating and/or preventing tumor metastases is described, in which tumor-associated macrophages M2 (TAM-M2) are reprogrammed to tumor-associated macrophages M1 (TAM-M1).

In a second object, the present invention describes pharmaceutical preparations comprising one of the compounds described.

In particular, said preparations are administered intravenously.

According to a particular aspect of the invention, such preparations can comprise the compound of the invention, possibly in recombinant form, and one or more pharmaceutically acceptable carriers and/or excipients.

In preferred aspects of the invention, such compounds can optionally be conjugated to appropriate molecules having therapeutic activity.

Such molecules can be chosen from the group of molecules used in the treatment of the specific form of tumor.

More in detail, such preparations are formulated for intravenous administration.

In a further object, the present invention describes a method for the treatment and/or prevention of tumor metastases comprising the administration to a subject in need of a pharmaceutically effective quantity of the compound of the invention.

In particular, such method includes the reprogramming of tumor-associated M2 macrophages (TAM-M2) to M1 tumor-associated macrophages (TAM-M1).

In one aspect of the invention, the production of IL12 is induced in M2-type macrophages.

This induction is greater than IL12 production in M1-type macrophages.

### Example 1

### Effect of ABRβ1 on IL10 Production in Macrophages

### Isolation of Human Monocytes

To prepare the monocytes from human peripheral blood, collect 2-4 bags of fresh plasma (buffy coat) and transfer into 50 ml sterile tubes. Centrifuge at 3000×g for 15 minutes at room temperature to remove the residual platelets and gather all the supernatants. Prepare the 50 ml falcons with 15 ml of Ficoll solution (1.077 g/ml) on the bottom, at room temperature. For each falcon, very gently deposit 30-35 ml of buffy coat on the Ficoll solution to create the first density gradient. Centrifuge at 400×g for 30 min at room temperature with very slow deceleration to prevent re-mixing. Collect the white ring of peripheral blood mononuclear cells (PBMC) which forms between the two phases in each 50 ml falcon and transfer it to a new sterile container. At this point add an excess of PBS-EDTA (1 mM) to the collected cells and centrifuge at 300×g for 10 min at room temperature. Remove the supernatant and repeat the procedure to wash the collected cells. At this point, resuspend the washed cell pellets in RPMI-1640 medium without phenol red added with 10% complement-inactivated FCS. Meanwhile, prepare the iso-osmotic Percoll solution for the second density gradient. To this end, mix the solutions in the following ratio: 23.13 ml of Percoll solution (density 1.131 g/ml) with 1.87 ml of 10×PBS. Transfer 23 ml of this solution into a 50 ml tube and add 27 ml of RPMI-1640 with phenol red added with complement-inactivated FCS (final 10%). All operations are carried out at room temperature. Place 25 ml of the freshly prepared solution in a 50 ml tube and deposit the PBMCs above such a solution, very slowly so as to avoid re-mixing. At this point centrifuge at 550×g without for 30 min at room temperature and slow down very slowly. Gently collect the white ring of monocytes located between the two phases of different density and transfer it into a new 50 ml tube, add PBS-EDTA (1 mM) and centrifuge at 400×g for 10 min at room temperature. Remove the supernatant and resuspend the cell pellet in RPMI-1640 medium with phenol red supplemented with de-complemented FCS to reach the final concentration of 10%.

### In Vitro Culture and Differentiation of Human Monocytes

Firstly, it is necessary to determine the concentration of monocytes in the resuspension solution after the isolation from human plasma, whereby the resuspended cells are counted using the vital dye trypan blue. The monocytes are then seeded in culture dishes using medium consisting of RPMI-1640, 2% AB human serum and 1% penicillin/streptomycin. The culture is incubated at 37° C. in 5% CO incubator for a few days before causing differentiation. For this purpose, the monocyte cultures are incubated for 10 days in the presence of 2 ng/ml GM-CSF or 2 ng/ml M-CSF. GM-CSF causes monocytes to differentiate into M1-type macrophages (anti-tumor) while M-CSF causes monocytes to differentiate into M2-type macrophages (pro-tumor).

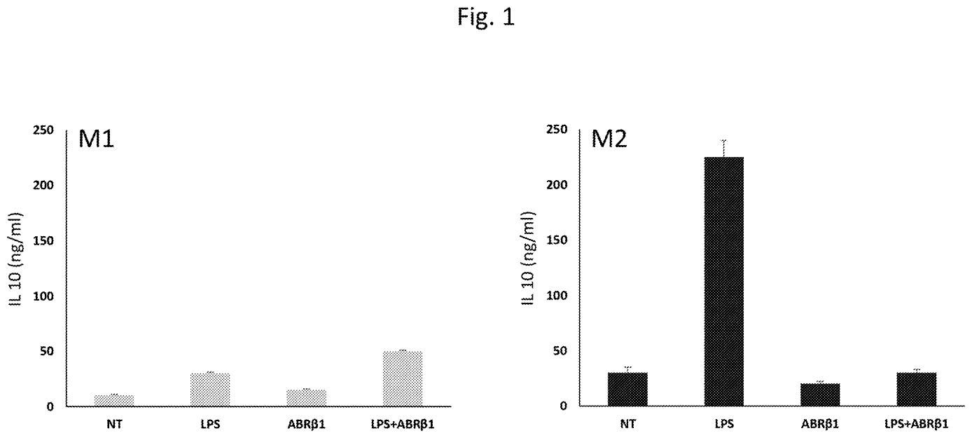

FIG. 1 shows that M2-type macrophages (differentiated in vitro) produce, unlike the M1-type macrophages, IL10 when appropriately stimulated with lipopolysaccharide (LPS). The treatment with the compound ABRβ1 causes a change in the phenotype of the macrophage M2 which loses the ability to produce IL10 if stimulated. This feature is instead typical of type-M1 macrophages.

### Example 2

### Effect of ABRβ1 on IL12 Production in Human Macrophages

To determine the type of effect of the compound ABRβ1 on the phenotype of human macrophages differentiated in vitro, the production of IL12, which characterizes M1-type macrophages, and the production of IL10, which is typically produced by M2-type macrophages, were analyzed. The above-mentioned interleukins were measured in the cell culture media following the different treatments using specific ELISA kits.

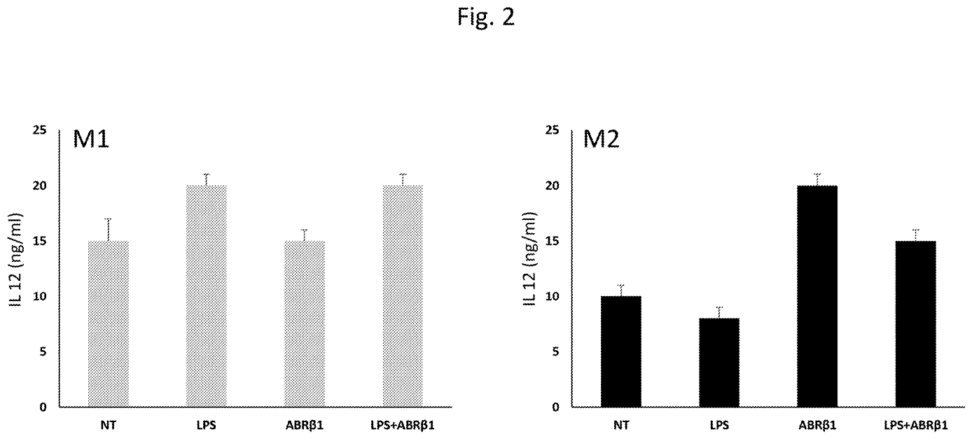

FIG. 2 shows that the M1-type macrophages produce IL12. The compound ABRβ1 shows a modest effect on the production of IL12 by the M1-type macrophages. In contrast, the compound ABRβ1 significantly stimulates the production of IL12 in M2-type macrophages, which are normally unable to do this. The data clearly demonstrate that the compound ABRβ1 is capable of reprogramming the M2-type macrophages, in particular, making them acquire the features of the M1 macrophages.

### Example 3

### Effect of ABRβ1 on Survival in a Syngeneic Mouse Model of Metastatic Melanoma

To evaluate the effect of immuno-stimulation and anti-metastatic activity of the compound ABRβ1 in vivo, a syngeneic mouse model of melanoma was chosen. The immune system of the mice receiving the tumor cells is perfectly functional, the murine cell line used is B16F10, characterized by high aggressiveness and high metastasis rate. The animals usually die in this mouse model due to metastases which originate rapidly at the level of the peritoneum or lungs. To this end, B16F10 cells were inoculated into the animal subcutaneously. Two experimental groups were set up: one control group (not treated with ABRβ1) and one group treated with ABRβ1. The compound was administered to the animals intraperitoneally at a dosage of 2 mg/kg twice a week. The treatment was started immediately after inoculation of the tumor cells in the animal. Survival analysis was carried out using the Kaplan-Meier model.

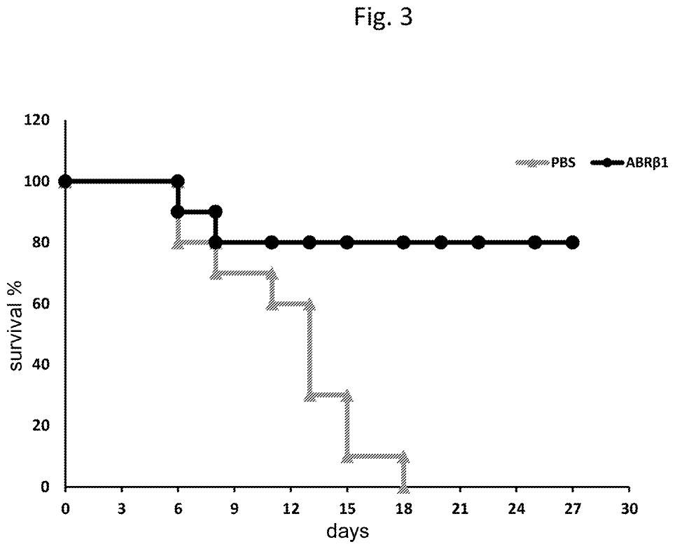

FIG. 3 shows that the mice inoculated with B16F10 cells did not survive beyond 18 days. In contrast, the mice inoculated with the tumor cells and treated with the compound ABRβ1 showed high survival (80%) well beyond 18 days. Surprisingly, upon the animals' sacrifice, the necropsy analysis showed no presence of metastases at the level of the peritoneum or lungs. The necroscopic analysis on the dead animals not treated with ABRβ1 instead showed the presence of widespread metastases in the peritoneal cavity and at the level of the lungs. This evidence clearly demonstrates that the compound ABRβ1 has marked anti-metastatic activity even in a particularly aggressive tumor model such as melanoma.

From the above description, the benefits offered by present invention will be apparent to those skilled in the art.

Of particular relevance is that the subunit of the invention has shown to have a clear and marked anti-metastatic activity, as shown by the highly effective data obtained on the metastatic mouse model of melanoma, which represents one of the most aggressive metastatic models.

Moreover, the use of the subunit ABRβ1 offers high quality and biological safety, by virtue of the production process which is almost free of risks of contamination from viruses, oncogenes, prions, toxins or residues of dangerous reagents normally used in the production of therapeutic proteins.

What is claimed is:

1. A method for treatment and/or prevention of tumor metastases in a subject in need thereof, the method comprising administering to the subject a pharmaceutically effective amount of a compound having an amino acid sequence corresponding to SEQ. ID NO: 1 or SEQ. ID NO: 2.

2. The method of claim 1, wherein said tumor metastases originate from a tumor selected from the group consisting of: melanoma, breast, ovary, pancreas, kidney, stomach, lung tumors, sarcomas, and neuroblastoma.

3. The method of claim 1, wherein said tumor metastases are lung, peritoneum, brain, kidney, or liver metastases.

4. The method of claim 1, wherein said treatment and/or prevention occurs by stimulating the immune system.

5. The method of claim 1, wherein tumor-associated macrophages M2 (TAM-M2) are reprogrammed to tumor-associated macrophages M1 (TAM-M1).

6. The method of claim 1, wherein said compound is obtained by a process comprising the steps of:
I) obtaining an appropriate vector transformed with a plasmid comprising the sequence corresponding to SEQ. ID NO:3;
II) transforming plant cells with the vector of step I);
III) selecting the transformed plant cells;
IV) culturing stable plant cells in suspension;
V) preparing a cell extract; and
VI) purifying the compound.

7. The method of claim 6, wherein in step II) plant cells of Nicotiana benthamiana are transformed.

8. The method of claim 6, wherein said compound is represented by the sequence corresponding to SEQ. ID NO: 2.

9. (canceled)

10. (canceled)

11. The method of claim 1, wherein a production of IL12 is induced in M2-type macrophages.

12. The method of claim 11, wherein the production of IL12 induced in the M2-type macrophages is higher than a production of IL12 in M1-type macrophages.


SEQUENCE LISTING (3 sequences)

SEQ ID NO: 1 - protein, 111 aa, Homo sapiens
NSCELTNITIAIEKEECRFCISINTTWCAGYCYTRDLVYKDPARPKIQKTCTFKELVYETVRVPGCAHHADSLYTYPVATQCHCGKCDSDSTDCTVRGLGPSYCSFGEMKE

SEQ ID NO: 2 - protein, 121 aa, synthetic construct
  REGION 1..121: note = compound
HHHHHHNSCELTNITIAIEKEECRFCISINTTWCAGYCYTRDLVYKDPARPKIQKTCTFKELVYETVRVPGCAHHADSLYTYPVATQCHCGKCDSDSTDCTVRGLGPSYCSFGEMKEKDEL

SEQ ID NO: 3 - DNA, 472 bp, synthetic construct
  misc_feature 1..472: note = encoding SEQ. ID. n. 2
  source 1..472: mol_type = other DNA
gaattcaacaatggctactcagagaagggctaacccatcttctcttcacctgattaccgtgttctctctgcttgtggctgtggtgtctgctgaggtgttccatcatcaccatcatcacaattcttgcgagctgaccaacatcaccattgctatcgagaaagaagagtgcaggttctgcatcagcatcaacactacttggtgcgctggttactgctacaccagggatcttgtgtacaaggatcctgctaggcctaagatccaaaagacctgcaccttcaaagagctggtttacgagactgttagggtgccaggttgtgctcatcatgctgattctctgtacacctaccctgttgctactcagtgccattgcggtaagtgcgatagcgattctactgattgcaccgtgagaggtctgggaccttcttactgttctttcggtgagatgaaagaaaaggatgagctgtagtctaga